(19) United States
(12) Patent Application Publication
(10) Pub. No.: US 2008/0147186 A1
(43) Pub. Date: Jun. 19, 2008

(54) Electrochemical Implant For Delivering Beneficial Agents

(76) Inventors: Ashok V. Joshi, Salt Lake City, UT (US); Akash Akash, Salt Lake City, UT (US)

Correspondence Address: CERAMATEC, INC., 2425 SOUTH 900 WEST, SALT LAKE CITY, UT, 84119, US

(21) Appl. No.: 11/611,070

(22) Filed: Dec. 14, 2006

(51) Int. Cl. A61F 2/02; A61N 1/00

(52) U.S. Cl. 623/11.11; 607/2; 607/115

(57) ABSTRACT

An apparatus for providing beneficial agents to the body is disclosed in one aspect of the invention as including an implant and a device integrated with the implant to generate a beneficial agent, such as iodine, chlorine, or other halogens. The device includes electrodes to conduct an electrical current and a substantially solid layer between the electrodes. An electrical current passes between the electrodes to electrochemically generate the beneficial agent. The implant may include a variety of devices to produce the beneficial agent, including for example an electrochemical cell, a capacitor, an electrochemical capacitor, a galvanic cell, or the like. Similarly, because of the solid state construction of the device, the device may, in certain embodiments, be incorporated into a load-bearing implant. This may be useful for use with certain types of implants, such as orthopedic implants.

### BACKGROUND OF THE INVENTION

1. Field of the Invention

The present invention relates to medical implants, and more particularly to electrochemical implants capable of delivering beneficial agents to the body.

2. Background

A medical implant is an artificial device used to replace a missing or damaged biological structure. Medical implants may encompass a wide variety of devices, including but not limited to soft tissue implants, orthopedic implants, or cardiovascular implants. Some implants, such as artificial pacemakers or cochlear implants, contain sophisticated electronics. Other implants include compound structures or act as reinforcement for various biological structures, such as dental implants or knee joint replacement implants. Yet other implants provide beneficial agents to the body, such as drug-eluting stents inserted into the aorta or coronary arteries.

Because most implants act as a magnet to bacteria, infection is one of the primary causes of implant failure and may result in prolonged pain and medical expense. While infections from contamination during surgery are rare, bacteria often spread to implants from infections in other parts of the body. When an implant becomes infected, a physician must often replace the implant and administer an aggressive regimen of antibiotics to kill the bacteria. This procedure often costs tens of thousands of dollars and causes extensive pain and recovery time.

Although bacterial growth may often be prevented by cleaning a surface with a disinfectant or treating the body with antibiotics, bacteria often irreversibly adheres to both artificial and natural surfaces that are surrounded by body fluids. Once the bacteria adhere, they can multiply to form complex multilayered colonies. These colonies produce a slimy matrix material, called a biofilm, which coats and protects the bacterial cells. This biofilm is difficult and often impossible to eliminate from the body with antibiotics because of the physical and chemical barrier it creates.

Due to the difficulty of eradicating infection, it is preferable to prevent infection from starting altogether. Currently, when an orthopedic implant is inserted into the body, a physician may apply an antibiotic-bearing adhesive to the site to prevent infection. The goal of the adhesive is to protect the implant and strengthen its attachment to the bone. However, these adhesives are not always reliable and the implant often becomes infected at a later time.

In view of the foregoing, what are needed are improved apparatus and methods for delivering beneficial agents, such as anti-infective agents, to areas of the body, including areas immediately around an implant. Such apparatus and methods would ideally be suitable for both load-bearing and non-load-bearing implants.

### SUMMARY OF THE INVENTION

Consistent with the foregoing, and in accordance with the invention as embodied and broadly described herein, an apparatus for providing beneficial agents to the body is disclosed in one aspect of the invention as including an implant and a device integrated with the implant to generate a beneficial agent, such as iodine, chlorine, or other halogens. The device includes electrodes to conduct an electrical current and a substantially solid layer between the electrodes. An electrical current passes between the electrodes to electrochemically generate the beneficial agent. The implant may include a variety of devices to produce the beneficial agent, including for example an electrochemical cell, a capacitor, an electrochemical capacitor, a galvanic cell, or the like. Similarly, because of the solid state construction of the device, the device may, in certain embodiments, be incorporated into a load-bearing implant. This may be useful for use with certain types of implants, such as orthopedic implants.

In another aspect of the invention, a method for providing beneficial agents to the body may include providing an implant and electrochemically generating a beneficial agent. This beneficial agent may be produced by generating an electrical current between electrodes, separated by a substantially solid layer, incorporated into the implant. In selected embodiments, the method may include electrochemically generating the beneficial agent prior to inserting the implant into the body. In other embodiments, the method may include electrochemically generating the beneficial agent after inserting the implant into the body. Similarly, the beneficial agent may be generated from compounds in the implant, or alternatively, by conducting an electrical current through body fluids of the implantee.

In another aspect of the invention, an apparatus for providing beneficial agents to the body includes an implant and an electrochemical cell integrated with the implant to generate a beneficial agent. The electrochemical cell includes a layer containing a beneficial agent chemically bound within a compound. Electrodes are placed in contact with each side of the layer to conduct an electrical current and create opposite charges on the electrodes. These opposite charges break down the compound to release the beneficial agent. An electrolyte layer is provided to transport ions between the electrodes.

In another aspect of the invention, an apparatus for providing beneficial agents to the body includes an implant and an electrochemical capacitor integrated with the implant to generate a beneficial agent. The electrochemical capacitor includes electrodes to conduct an electrical current therebetween and store opposite electrical charges. At least one of the electrodes contains a beneficial agent that is chemically bound within a compound. This beneficial agent is released from the compound by storing opposite electrical charges on the electrodes. In selected embodiments, the electrolyte layer is solid or substantially solid so the electrochemical capacitor may be used in load-bearing applications.

In another aspect of the invention, an apparatus for providing beneficial agents to the body includes an implant and a capacitor integrated with the implant to generate a beneficial agent. The capacitor includes a pair of electrodes to conduct an electrical current, store opposite electrical charges, and discharge the charges through body fluids of the implantee. This electrical current ionizes or breaks down certain compounds, such as sodium chloride, in the body fluids. A dielectric layer is placed between the electrodes. In certain embodiments, the dielectric layer is constructed of a substantially solid material.

In yet another aspect of the invention, an apparatus for providing beneficial agents to the body includes an implant and a galvanic cell integrated into the implant to generate a beneficial agent. The galvanic cell includes electrodes to discharge an electrical current through body fluids of the implantee to generate a beneficial agent. An electrolyte layer is placed between the electrodes to provide an ion transport mechanism therebetween.

### BRIEF DESCRIPTION OF THE DRAWINGS

In order to describe the manner in which the above-recited features and advantages of the present invention are obtained, a more particular description of apparatus and methods in accordance with the invention will be rendered by reference to specific embodiments thereof, which are illustrated in the appended drawings. Understanding that these drawings depict only typical embodiments of the present invention and are not, therefore, to be considered as limiting the scope of the invention, apparatus and methods in accordance with the present invention will be described and explained with additional specificity and detail through the use of the accompanying drawings in which:

FIG. 1 is a high-level schematic block diagram of one embodiment of an implant in accordance with the invention incorporating an electrochemical cell;

FIG. 2 is a high-level schematic block diagram of an embodiment of an implant incorporating an electrochemical capacitor.

FIG. 3 is a high-level schematic block diagram of an embodiment of an implant incorporating a capacitor;

FIG. 4 is a high-level schematic block diagram of an embodiment of an implant incorporating a galvanic cell;

FIG. 5 is a high-level schematic block diagram of one embodiment of an implant infiltrated with a material used to generate a beneficial agent.

FIG. 6 is a high-level schematic block diagram of one embodiment of a multi-layered implant;

FIG. 7 is a high-level schematic block diagram of one embodiment of an implant incorporating one or more micro-channels for disbursing a beneficial agent; and

FIG. 8 is a high-level schematic block diagram of another embodiment of an implant in accordance with the invention.

### DETAILED DESCRIPTION OF THE INVENTION

It will be readily understood that the components of the present invention, as generally described and illustrated in the Figures herein, could be arranged and designed in a wide variety of different configurations. Thus, the following more detailed description of the embodiments of apparatus and methods in accordance with the present invention, as represented in the Figures, is not intended to limit the scope of the invention, as claimed, but is merely representative of certain examples of presently contemplated embodiments in accordance with the invention. The presently described embodiments will be best understood by reference to the drawings, wherein like parts are designated by like numerals throughout.

For the purposes of this specification, the phrase “substantially solid” is used to describe materials or composites that are solid or nearly solid.

FIGS. 1 though 8 show various embodiments of an implant 10 in accordance with the invention. One of skill in the art will recognize that a medical implant 10 may take on a wide variety of shapes and configurations. Thus, the implant 10 illustrated in FIGS. 1 through 8 is illustrated at a high level to simplify and provide a general understanding of the invention. Consequently, the principles taught in association with FIGS. 1 though 8 may be applied to a wide variety of medical implants 10 having diverse shapes, sizes, purposes, and configurations, including but not limited to soft tissue, orthopedic, cardiovascular, or other types of implants. This includes beads, discs, cylinders, or other shapes used to replace bones or refill cavities. Furthermore, features of certain embodiments of the invention may be readily applied to other embodiments of the invention illustrated herein.

Referring to FIG. 1, in one embodiment, an implant 10 in accordance with the invention may include a pair of electrodes 12a, 12b and an electrolyte layer 14 between the electrodes 12a, 12b. The electrodes 12a, 12b and layer 14 together form an electrochemical cell 16. The electrochemical cell 16 may itself form the implant 10 (as illustrated) or the cell 16 may be integrated into or contained within an implant 10. This also applies to each of the embodiments illustrated in FIGS. 1 through 8. The electrodes 12a, 12b may be constructed of an electrically conductive material that is also biocompatible, which may include, for example metal, carbon, or composites of metal or carbon, including but not limited to iron, cobalt, chromium, titanium, tantalum, and alloys thereof.

As will be explained in more detail hereafter, in selected embodiments, the electrodes 12a, 12b may be constructed of a porous material, to enable bone and tissue to grow into and attach to the implant 10, as well as to enable the diffusion of beneficial agents through the electrodes 12a, 12b. In other embodiments, the outer surface of the electrodes 12a, 12b and electrolyte layer 14 may be roughened or textured to enable bone or other tissue to grow into and/or grip the implant 10. In selected embodiments, the electrodes 12a, 12b and/or the electrolyte layer 14 may be formed from powders which are sintered together to form a solid state electrochemical cell 16.

The electrolyte layer 14 may include a compound containing a beneficial agent. This beneficial agent may be chemically bound within the compound and may be released by breaking down, or disassociating, the compound. To release the beneficial agent, an electrical current may be generated across the electrodes 12a, 12b to create opposite electrical charges on the electrodes 12a, 12b. These charges break down the compound to release the beneficial agent. In certain embodiments, a voltage source 18 may be applied to the electrodes 12a, 12b prior to implantation to release the beneficial agent.

For example, in one embodiment, the electrolyte layer 14 may contain a metal halide, such as a silver halide (e.g., silver bromide (AgBr), silver chloride (AgCl), silver iodide (AgI), etc.), which may be represented by the notation AgX. In certain embodiments, the silver halide may be combined or mixed with a ceramic material to create a composite (which may be represented as the composite AgX+C, where C represents a ceramic material) to give the layer 14 additional strength and/or ionic conductivity. Suitable ceramics may include, for example, aluminum oxide (Al₂O₃), silicon carbide (SiC), zirconium oxide (ZrO₂), or the like. The added strength may be particularly useful in load-bearing implant applications, such as orthopedic implants (e.g., knee, hip, spinal implants, etc.), where strength may be an important factor. This may also enable the electrochemical cell 16 to absorb all or part of the stresses exerted on the implant 10. This provides a significant advantage over solution-based or non-solid state electrochemical cells, which may be unable to bear a significant load. Moreover, this may also be considerably safer than solution-based or non-solid state electrochemical cells which may rupture and spill their contents into the implantee, potentially causing injury or even death.

Consider one embodiment of an electrolyte layer 14 containing silver iodide (AgI), which will dissociate at approximately 0.8 volts. Upon applying a voltage of at least 0.8 volts and generating an electrical current between the electrodes 12a, 12b, opposite charges accumulate on each of the electrodes 12a, 12b. For example, positive charges accumulate on the electrode 12a and negative charges accumulate on the electrode 12b. These charges ionize (i.e., break down) the silver iodide (AgI) into constituent silver ions (Ag⁺) 20 and iodide ions (I⁻) 22. The silver ions 20 are attracted to and accumulate at the interface 24 where they combine with available electrons on the electrode 12b. Similarly, the iodide ions 22 are attracted to and accumulate at the interface 26, where they combine with positive charges on the electrode 12a.

Because of the high vapor pressure of iodine at body temperature, the iodine will tend to diffuse through the porous electrode 12a into the body of the implantee. The silver, on the other hand will tend to remain within the implant 10 at the interface 24. The iodine release rate may depend on factors such as the pore size of the electrode 12a and the amount of iodine 22 released upon breaking down the compound in the layer 14. This may be adjusted by changing the voltage 18 or the amount of time the voltage 18 is applied to the electrodes 12a, 12b. Once dispersed, the iodine 22 is effective to kill bacteria or prevent infection from starting around the implant 10.

One of ordinary skill in the art will recognize that in addition to iodine, other beneficial agents such as chlorine, bromine, or the like, may be electrochemically generated in a similar manner to that described above. Thus, all beneficial agents generated by an implant 10 using the apparatus or methods described herein are encompassed within the scope of the present invention.

As previously mentioned, in selected embodiments, a voltage source 18 may be applied to the electrodes 12a, 12b prior to implanting the implant 10 to release the beneficial agent. For example, a surgeon may apply a voltage to the implant 10 immediately prior to insertion into the implantee. The surgeon may then remove the implant 10 from the voltage 18 and insert the implant 10 into the implantee. Once the beneficial agent is released, the beneficial agent will begin to diffuse through the implant 10 to provide various benefits, such as killing bacteria or preventing infections form occurring. In other embodiments, it is contemplated that a voltage source 18, such as a battery, capacitor, or the like, could be installed in the implant 10 to generate the beneficial agent.

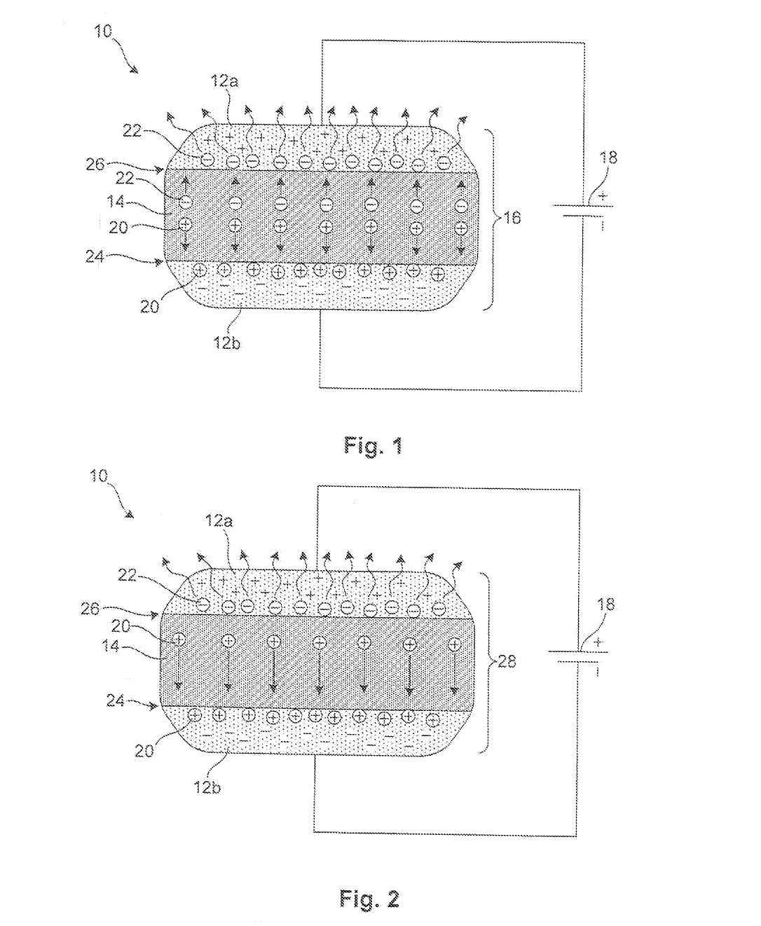

Referring to FIG. 2, in another embodiment, an implant 10 in accordance with the invention may include a pair of electrodes 12a, 12b and an electrolyte layer 14 between the electrodes 12a, 12b. The electrodes 12a, 12b and electrolyte layer 14 together form an electrochemical capacitor 28. The electrodes 12a, 12b may be constructed of a composite containing a porous electrically conductive material and a compound containing a beneficial agent. Like the previous example, the beneficial agent may be chemically bound within the compound. To release the beneficial agent, a voltage 18 may be applied across the electrodes 12a, 12b to create opposite electrical charges on the electrodes 12a, 12b. These charges break down the compound in the electrodes 12a, 12b to release the beneficial agent.

For example, in one embodiment, an electrode 12a may include a composite containing a compound, such as silver iodide, mixed with an electrically conductive material (e.g., carbon). When a voltage is applied across the electrodes 12a, 12b, the electrodes 12a, 12b accumulate opposite electrical charges. Positive charges accumulate on the electrode 12a and negative charges accumulate on the electrode 12b. The negatively charged electrode 12b attracts the positively charged silver ions of the silver iodide compound in the electrode 12a. This breaks the chemical bond between the silver ions (Ag⁺) 20 and iodide ions (I⁻) 22. The silver ions 20 are conducted through the electrolyte layer 14 until they reach the electrode 12b and combine with available electrons. The iodide ions 22, in contrast, remain at the electrode 12a where they combine with positive charges and dissipate through the pores of the electrode 12a.

To conduct the silver ions between the two electrodes 12a, 12b, the electrolyte layer 14 includes a silver ion conductor, such as silver iodide, silver oxide, silver bromide, or the like, which is inert to the iodide ions 22. The electrolyte layer 14 may be formed as a solid or substantially solid layer 14 in order to utilize the electrochemical capacitor 28 in load-bearing applications and to prevent or reduce the safety risk associated with liquid electrolytes.

One of ordinary skill in the art will recognize that other beneficial agents may be produced using an electrochemical capacitor 28 in accordance with the invention. Thus, an implant 10 containing an electrochemical capacitor 28 producing other types of beneficial agents is intended to be captured within the scope of the present invention. For example, in other embodiments, an electrode 12a may contain the compound sodium chloride (NaCl). This compound may be broken down into sodium ions (Na⁺) and chlorine ions (Cl⁻) with charges on the electrodes 12a, 12b in a similar manner to breaking down silver iodide. The sodium ions may be conducted through the electrolyte layer 14 and the chlorine ions may be dissipated through the porous electrode 12a. Chlorine, like iodine, exhibits rapid and effective anti-infective action even in small quantities.

Like the previous example, a voltage 18 may be applied to the electrodes 12a, 12b of the electrochemical capacitor 28 prior to inserting the implant 10 to charge the capacitor 28 and release the beneficial agent. The implant 10 may then be removed from the voltage 18 and inserted into the patient. Once the beneficial agent is released, the beneficial agent may diffuse through the electrode 12a at a rate determined by factors such as pore size and density in the electrode 12a, and the quantity of beneficial agent generated by the capacitor 28.

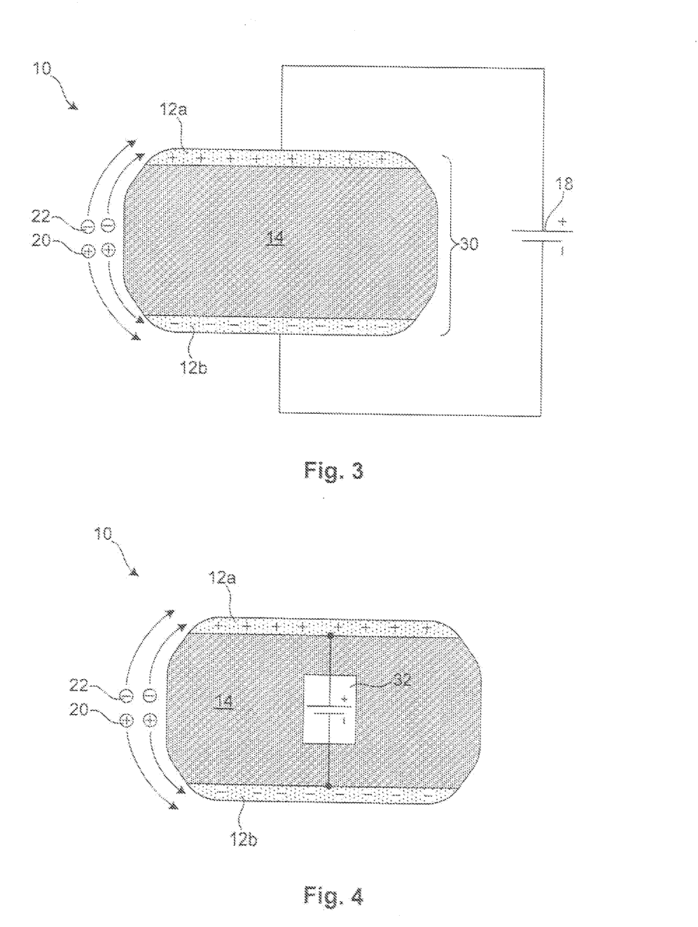

Referring to FIG. 3, in another embodiment, an implant 10 in accordance with the invention may include a pair of electrodes 12a, 12b separated by a dielectric layer 14. These components 12a, 12b, 14 together form a capacitor 30. The electrodes 12a, 12b may function as the “plates” of the capacitor 30 and be constructed of an electrically conductive material such as metal, carbon, or alloys or composites thereof. In selected embodiments, the electrodes 12a, 12b, may simply be a conductive coating on each side of the dielectric layer 14. The dielectric layer 14 may include materials such as aluminum oxide (Al₂O₃) or zirconium oxide (ZrO₂).

Upon applying a voltage 18, opposite electrical charges are stored on the electrodes 12a, 12b. When the implant 10 is inserted into the body, these charges discharge over time (e.g., hours, days, months, etc., depending on the design of the capacitor) through the body fluids of the implantee to generate a beneficial agent.

For example, in one embodiment, the capacitor 30 may produce chlorine by breaking down sodium chloride (NaCl) in the body fluids of an implantee. In this example, an electrical current is generated between a negatively charged electrode 12b, or cathode 12b, and a positively charged electrode 12a, or anode 12a. The electrical current splits the sodium chloride and the positively charged sodium ions (Na⁺) 20 move towards the cathode 12b and the negatively charged chlorine ions (Cl⁻) 22 move towards the anode 12a. The voltage needed to separate the ions 20, 22, which is approximately three volts for sodium chloride, is provided by the charge stored in the capacitor 30. The chemical reaction of an aqueous solution of sodium chloride may be described as follows:

2NaCL+2H₂O→Cl₂+H₂+2NaOH

Because excessive doses of chlorine may be harmful to the body for many of the same reasons that chlorine destroys bacteria and other microorganisms, the chlorine generation rate may depend on factors such as the voltage of the capacitor and the amount of charge stored on each electrode 12a, 12b. Ideally, the implant 10 is designed to generate a small enough dose to kill bacterial locally around the implant 10 without causing harm to the body.

In practice, a voltage source 18 may be applied to the electrodes 12a, 12b to charge the capacitor 30 prior to implantation. The implant 10 may then be removed from the voltage source 18 and implanted into the patient. The capacitor 30 may then be discharged through the body fluids of the implantee to produce a beneficial agent until the charge is depleted.

In another contemplated embodiment of the invention, a pair of electrodes 12a, 12b, without a dielectric layer 14, may provide a corona generator. This generator may utilize charges on the electrodes 12a, 12b to generate an arc between the electrodes 12a, 12b. This arc may be used to produce a beneficial agent such as ozone (O₃), which is an effective anti-microbial agent.

Referring to FIG. 4, in another embodiment, an implant 10 in accordance with the invention may include a pair of electrodes 12a, 12b connected to a voltage source 32, such as a galvanic cell 32. The galvanic cell 32 may be contained within the implant 10 and may, in certain embodiments, be encased within a protective housing such as titanium. Like the capacitor 30 discussed in association with FIG. 3, the galvanic cell 32 may be used to produce a beneficial agent in the body by generating an electrical current through body fluids. In one embodiment, this current may be used to break down a compound, such as sodium chloride (NaCl), to produce an anti-infective agent.

Like the previous example, the chlorine generation rate may depend on factors such as the voltage of the galvanic cell 32, the internal resistance of the cell 32, as well as the resistance of body fluids conducting the current. The electrical current may also be adjusted with a resistor. In this way, the current may be adjusted to produce small doses of chlorine without having a detrimental effect on the body. Furthermore, a galvanic cell 32 may have a significantly longer life than a capacitor. This may be desirable for implants 10 providing sustained release of beneficial agents over the course of days, months, or even years.

In certain embodiments, the components of the galvanic cell 32, including the anode, cathode, and electrolyte, may be constructed entirely of solid state components. This may increase the safety of the galvanic cell 32 and enable the galvanic cell 32 to bear a load. Alternatively, instead of providing a galvanic cell 32 inside the implant 10, the implant itself may be a galvanic cell 32. For example, the galvanic cell 32 may include an anode 12a, a cathode 12b, separated by a solid or substantially solid electrolyte layer 14. The anode 12a may oxidize to release electrons and the cathode 12b may be reduced by receiving electrons. The electrolyte layer 14 may provide an ion transport mechanism between the anode 12a and cathode 12b.

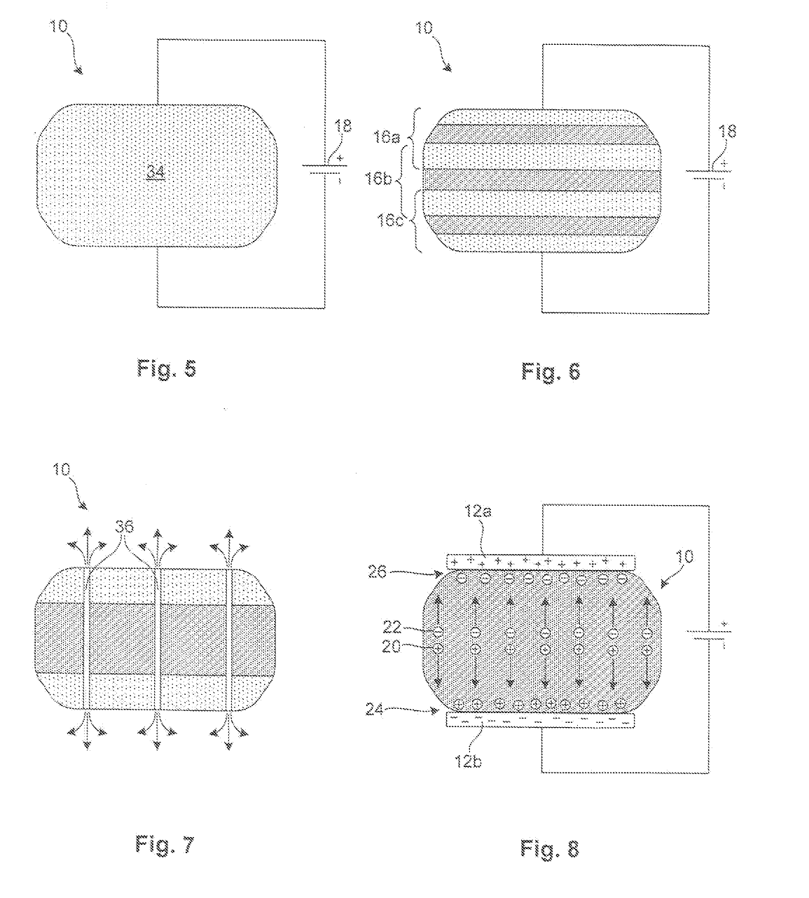

Referring to FIG. 5, in selected embodiments, an implant 10 may include an electrically conductive porous material 34, such as porous titanium, infiltrated with a compound containing a beneficial agent. Prior to implantation, a voltage source 18 may be applied to the material 34 to break down the compound and release the beneficial agent. Once the implant 10 is inserted in to the body, the beneficial agent may seep or dissipate through the pores of the material 34 to disinfect or provide other benefits around the implant 10.

For example, the porous material 34 may be infiltrated with a compound such as silver iodide (AgI). Prior to implantation, a voltage source 18 may be applied to the material 34 to generate a current therethrough. The current may be sufficient to break down and disassociate some or all of the silver iodide into silver ions (Ag⁺) and iodide ions (I⁻). Due to the high vapor pressure of iodine at body temperature, the iodine will tend to diffuse through the porous material 34. In contrast, the silver will tend to remain inside the implant 10. As in other embodiments, the iodine release rate may depend upon factors such as the pore size and pore density of the material 34, the amount of iodine generated within the implant 10, and the like.

Referring to FIG. 6, in certain embodiments, an implant 10 may include a multi-layer structure. For example, two or more electrochemical cells 16, electrochemical capacitors 28, capacitors 30, or galvanic cells 32, as described in association with FIGS. 1 through 4, may be combined in series or parallel in a single device 10. In selected embodiments, different devices 16, 28, 30, 32, 34 may be combined in a single implant 10. These devices 16, 28, 30, 32, 34 may produce either the same or different beneficial agents.

For example, a multi-layered implant 10 could include one, two, or even three electrochemical cells 16a, 16b, 16c combined into a single device. A voltage source 18 may be applied to the multi-layered implant 10 to release the beneficial agent or agents produced by each of the three cells 16a, 16b, 16c. Because a voltage drop may occur across each of the cells 16a, 16b, 16c, the voltage source 18 may need to be increased to apply sufficient voltage 18 across each of the cells 16a, 16b, 16c.

Referring to FIG. 7, in certain embodiments, micro-channels 36 may be incorporated into an implant 10 in accordance with the invention. These micro-channels 36 may contain beneficial agents, such as anti-inflammatory, pain relief, anti-infective, or other medications, and may also provide locations for bone or other tissue to grow into the implant 10. In selected embodiments, the beneficial agents may disperse from the micro-channels 36 when the implant 10 is inserted into the body and comes into contact with body fluids.

The micro-channels 36 may be incorporated into any of the implants 10 described in association with FIGS. 1 through 6 to provide additional beneficial agents. Furthermore, one of ordinary skill in the art will appreciate that micro-channels 36 in accordance with the invention may be provided in diverse numbers, sizes, shapes, and orientations in an implant 10, and may or may not extend all the way through the implant 10, without departing from the scope of the present invention.

Referring to FIG. 8, in another embodiment, an implant 10 in accordance with the invention may be constructed of a composite of one or more ceramics plus one or more halides, or a composite of one or more ceramics plus one or more peroxides. Suitable ceramics for inclusion in these composites may include, for example, metal oxides, metal carbides, metal nitrides, or combinations thereof, where the metal includes, for example, Al, Ti, Ta, Co, Fe, Nb, or combinations thereof. Similarly, suitable halides may include, for example, AgI, I₂, AgCl, AgBr, or combinations thereof. Suitable peroxides may include, for example, AgO (silver peroxide). The halide or peroxide component may contain a beneficial agent (e.g., iodine, chlorine, bromine, oxygen, etc.) whereas the ceramic material may be used to provide ionic conductivity and/or structural integrity to the implant 10, thereby making it robust enough for use in load-bearing applications such as orthopedic (e.g., bone-replacement) or spinal implants 10.

Prior to implantation, the implant 10 may be inserted between the electrodes 12a, 12b, where an electric current may be used to create opposite electrical charges on the electrodes 12a, 12b. These charges may break down the halide or peroxide components to release the beneficial agent (i.e., iodine, bromine, chlorine, oxygen, etc.) contained therein. For example, where the implant 10 is constructed of the composite of AgI+Al₂O₃, the charges on the electrodes 12a, 12b may ionize the silver iodide (AgI) into silver ions (Ag⁺) 20 and iodide ions (I⁺) 22, where they may accumulate at the interfaces 24, 26. Where, the implant 10 is constructed of, for example, the composite AgO+TiO₂, the charges on the electrodes 12a, 12b may be used to ionize the silver peroxide into silver ions 20 and oxygen ions 22 where they may accumulate at the interfaces 24, 26. Once the beneficial agents are released, the implant 10 may be removed from the electrodes 12a, 12b and inserted into the patient. Thus, in certain embodiments, the electrodes 12a, 12b may be separate and distinct from the implant 10.

The present invention may be embodied in other specific forms without departing from its essence or essential characteristics. The described embodiments are to be considered in all respects only as illustrative, and not restrictive. The scope of the invention is, therefore, indicated by the appended claims, rather than by the foregoing description. All changes within the meaning and range of equivalency of the claims are to be embraced within their scope.

We claim:

1. An apparatus comprising:
a load-bearing implant; and
a device integrated into the implant to generate a beneficial agent, the device comprising:
first and second electrodes to conduct an electrical current, the electrical current electrochemically generating a beneficial agent upon flowing between the first and second electrodes; and
a substantially solid layer interposed between the first and second electrodes.

2. The apparatus of claim 1, wherein the device is one of an electrochemical cell, a capacitor, an electrochemical capacitor, and a galvanic cell.

3. The apparatus of claim 1, wherein at least one of the first and second electrodes comprises a metal selected from the group consisting of iron, cobalt, chromium, titanium, tantalum and alloys thereof.

4. The apparatus of claim 3, wherein the metal is porous.

5. The apparatus of claim 1, wherein at least one of the first and second electrodes comprises a metal halide.

6. The apparatus of claim 5, wherein at least one of the first and second electrodes comprises a silver halide.

7. The apparatus of claim 1, wherein at least one of the first and second electrodes comprises a ceramic material.

8. The apparatus of claim 1 wherein the substantially solid layer is a dielectric material.

9. The apparatus of claim 1, wherein the substantially solid layer is an electrolyte.

10. The apparatus of claim 1, wherein the substantially solid layer comprises a metal halide.

11. The apparatus of claim 1, wherein the substantially solid layer comprises a silver halide.

12. The apparatus of claim 1, wherein the substantially solid layer comprises a ceramic material.

13. The apparatus of claim 1, wherein the beneficial agent is a halogen.

14. The apparatus of claim 1, wherein the device is an electrochemical cell and the first and second electrodes are porous.

15. A method for providing a beneficial agent with an implant, the method comprising:
providing an implant; and
electrochemically generating a beneficial agent by generating an electrical current between first and second electrodes incorporated into the implant, the first and second electrodes separated by a substantially solid layer.

16. The method of claim 15, wherein electrochemically generating comprises generating the beneficial agent using at least one of an electrochemical cell, a capacitor, an electrochemical capacitor, and a galvanic cell.

17. The method of claim 15, wherein electrochemically generating comprises generating the beneficial agent prior to inserting the implant into the body.

18. The method of claim 15, wherein electrochemically generating comprises generating the beneficial agent after inserting the implant into the body.

19. The method of claim 15, wherein electrochemically generating comprises conducting the electrical current through body fluids.

20. The method of claim 15, wherein at least one of the first and second electrodes comprises a porous metal;

21. The method of claim 20, further comprising disseminating the beneficial agent through the porous metal.

22. The method of claim 15, wherein at least one of the first and second electrodes comprises a metal halide.

23. The method of claim 22, wherein at least one of the first and second electrodes comprises a silver halide.

24. The method of claim 15, wherein at least one of the first and second electrodes comprises a ceramic material.

25. The method of claim 15, wherein the substantially solid layer is a dielectric material.

26. The method of claim 15, wherein the substantially solid layer is an ionically conductive ceramic material.

27. The method of claim 15, wherein the substantially solid layer comprises a metal halide.

28. The method of claim 15, wherein the substantially solid layer comprises a silver halide.

29. The method of claim 15, wherein the substantially solid layer comprises a ceramic material.

30. The method of claim 15, wherein providing an implant comprises providing one of a load-bearing implant, a bone-replacement implant, an orthopedic implant, and a spinal implant.

31. The method of claim 15, wherein the beneficial agent is a halogen.

32. An apparatus comprising:
an implant; and
an electrochemical cell integrated into the implant to generate a beneficial agent, the electrochemical cell comprising:
a layer comprising a beneficial agent chemically bound within a compound, the layer having a first side and a second side; and
a first electrode in contact with the first side and a second electrode in contact with the second side, the first and second electrodes conducting an electrical current to create opposite charges on the first and second electrodes, the opposite charges breaking down the compound to release the beneficial agent.

33. The apparatus of claim 32, wherein at least one of the first and second electrodes comprises a metal selected from the group consisting of iron, cobalt, chromium, titanium, tantalum, and alloys thereof.

34. The apparatus of claim 33, wherein the metal is porous.

35. The apparatus of claim 34, wherein the beneficial agent is dissipated through the porous metal.

36. The apparatus of claim 32, wherein the layer is an electrolyte.

37. The apparatus of claim 32, wherein the compound is a metal halide.

38. The apparatus of claim 37, wherein the compound is a silver halide.

39. The apparatus of claim 32, wherein the layer comprises a ceramic material.

40. The apparatus of claim 32, wherein the beneficial agent is a halogen.

41. The apparatus of claim 32, wherein the implant is one of a load-bearing implant, a bone-replacement implant, an orthopedic implant, and a spinal implant.

42. An apparatus comprising:
an implant; and
a capacitor integrated into the implant to generate a beneficial agent, the capacitor comprising:
first and second electrodes to conduct an electrical current and thereby store opposite electrical charges, wherein at least one of the first and second electrodes comprises a beneficial agent chemically bound within a compound, the beneficial agent being released from the compound by inducing opposite electrical charges on the first and second electrodes; and
an electrolyte layer to transport ions between the first and second electrodes.

43. The apparatus of claim 42, wherein the compound is a metal halide.

44. The apparatus of claim 43, wherein the compound is a silver halide.

45. The apparatus of claim 42, wherein at least one of the first and second electrodes comprises a ceramic material.

46. The apparatus of claim 42, wherein the electrolyte layer comprises a metal ion conductor.

47. The apparatus of claim 42, wherein the electrolyte layer comprises a ceramic material.

48. The apparatus of claim 42, wherein the beneficial agent is a halogen.

49. The apparatus of claim 42, wherein the implant is one of a load-bearing implant, a bone-replacement implant, an orthopedic implant, and a spinal implant.

50. The apparatus of claim 42, wherein the electrolyte layer is substantially solid.

51. An apparatus comprising:
an implant; and
a capacitor integrated into the implant to generate a beneficial agent, the capacitor comprising:
first and second electrodes to conduct an electrical current, store opposite electrical charges, and discharge through the body fluids of an implantee to release a beneficial agent from the body fluids; and
a dielectric layer between the first and second electrodes.

52. The apparatus of claim 51, wherein at least one of the first and second electrodes comprises a metal selected from the group consisting of iron, cobalt, chromium, titanium, tantalum and alloys thereof.

53. The apparatus of claim 52, wherein the metal is porous.

54. The apparatus of claim 51, wherein the dielectric layer comprises a ceramic material.

55. The apparatus of claim 51, wherein the beneficial agent is a halogen.

56. The apparatus of claim 51, wherein the implant is one of a load-bearing implant, a bone-replacement implant, an orthopedic implant, and a spinal implant.

57. The apparatus of claim 51, wherein the dielectric layer is substantially solid.

58. An apparatus comprising:
an implant; and
a galvanic cell integrated into the implant to generate a beneficial agent, the galvanic cell comprising:
first and second electrodes to discharge an electrical current through the body fluids of an implantee to release a beneficial agent from the body fluids; and
an electrolyte layer between the first and second electrodes.

59. The apparatus of claim 58, wherein at least one of the first and second electrodes comprises a metal selected from the group consisting of iron, cobalt, chromium, titanium, tantalum and alloys thereof.

60. The apparatus of claim 59, wherein the metal is porous.

61. The apparatus of claim 58, wherein the beneficial agent is a halogen.

62. The apparatus of claim 58, wherein the implant is one of a load-bearing implant, a bone-replacement implant, an orthopedic implant, and a spinal implant.

63. The apparatus of claim 58, wherein the electrolyte layer is substantially solid.

64. An orthopedic or spinal implant comprising a composite of a ceramic and a halide, wherein the ceramic comprises at least one of the group consisting of a metal oxide, a metal carbide, and a metal nitride, and wherein said metal comprises at least one of the group consisting of Al, Ti, Ta, Co, Fe, and Nb, and wherein said halide comprises at least one of the group consisting of AgI, I₂, AgCl, and AgBr.

65. A method for providing an orthopedic or spinal implant comprising:
providing an implant comprising a composite containing a ceramic and a halide; and
exposing the implant to an electrical potential to release a beneficial agent from the implant.

66. The method of claim 65, wherein exposing comprises exposing the implant to the electrical potential for up to one hour prior to inserting the implant into a recipient.

67. The method of claim 65, wherein the ceramic comprises a metal oxide and the halide comprises a silver halide.

68. A method for providing an orthopedic or spinal implant, the method comprising:
providing an implant comprising first and second electrodes, the implant providing beneficial agents upon activation; and
activating the implant by generating an electrical current between the first and second electrodes.
